(19) United States
(12) Patent Application Publication
(10) Pub. No.: US 2026/0348860 A1
(43) Pub. Date: Nov. 5, 2026

(54) POTASSIUM ION SELECTIVE OPTICAL SENSOR AND METHOD OF FABRICATION THEREOF

(71) Applicant: Vulcan Photonics Sdn. Bhd., Kuala Lumpur (MY)

(72) Inventors: Khong Nee KOO, Ayer Itam (MY); Mei Qun SEAH, Subang Jaya (MY); Farah Hidayah BINTI JAMALUDIN, Johor Bahru (MY)

(21) Appl. No.: 19/120,652

(22) PCT Filed: Oct. 12, 2022
(86) PCT No.: PCT/MY2022/050096

§ 371 Date: Apr. 11, 2025

(51) Int. Cl. G01N 21/77; A61B 5/145; G01N 21/17

(52) U.S. Cl. CPC G01N 21/7746; A61B 5/14546; G01N 2021/1789

(57) ABSTRACT

An ion-selective optical sensor for detecting potassium (K+) ions and a method for fabricating an ion-selective sensor comprises the following steps of: treating a surface of the sensor with (3-aminopropyl)triethoxysilane (APTES) followed by glutaraldehyde (GA); coating the APTES-GA surface with a crown ether ionophore to create a functionalized layer; wherein when the functionalized layer is in contact with the targeted ion in a sample, a complex is formed between the crown ether and the targeted ion therefore changing the material index of the functionalized layer, the change corresponding to the concentration of the targeted ion in the sample.

### FIELD OF INVENTION

The present invention generally relates to detection of metal cations, and more particularly to a sensor and method for fabricating a sensor for detecting and measuring the content of potassium (K+) ions in a sample.

### BACKGROUND

Measuring cations concentration have been of vital importance in various fields. For instance, quantifying the presence of cations plays a major role in the clinical and medical fields, since some cations are essential for many intracellular activities.

Potassium cations for instance, plays a significant role in the transmission of information by the nervous system and in the cycle of muscle tissue. Therefore, in the medical field, accurate, easy and rapid sensing of potassium ions is highly imperative, as these features can help to quickly assess the condition of a patient.

The choice of sensor depends on the type of ion concentration to be measured. Understandably, the sensor must be sensitive to activity of the targeted ion in preference to the activities of other ions which may be present in a sample or test solution. There are several modes of detecting the presence of ions in samples, one of which is the binding of targeted ions to chemical compounds, that usually leads to creation of strongly bound complexes indicative of the targeted ion's presence in the sample which can be used for quantification. It should be pointed out, however, that there are various conditions and variables that could affect cation recognition, stability and selectivity in such sensors, such as cavity dimensions, shape, substituent effect, conformational flexibility, type of donor atom and the solvent used.

There are several difficulties in measuring the content of cations such as sustainability and cost, as a great majority of the sensing devices and equipment requires sophisticated operational steps as well as complicated sample pre-processing process, which increases the fabrication cost in addition to being inappropriate for on-site or in situ measurements, considering most of these devices and equipment are laboratory-bound.

### SUMMARY

In one aspect, the present invention provides a method for forming an ion-selective optical sensor, the method comprising the steps of: treating a surface of the sensor with (3-aminopropyl)triethoxysilane (APTES) followed by glutaraldehyde (GA); and coating the APTES-GA surface with a crown ether-ionophore to create a functionalized layer; wherein when the ionophore functionalized layer is in contact with the targeted ion in a sample, a complex is formed between the crown ether and the targeted ion therefore changing the material index of the functionalized layer, the change corresponding to the concentration of the targeted ion in the sample.

Typically, the surface of the sensor comprises a resonant structure.

Typically, changing the material index shifts the resonant condition of the functionalized layer therefore enabling the measurement of ion concentration.

In an embodiment, the targeted ion is K+.

In a further embodiment, the method further includes a pre-treatment step to modify the surface of the sensor to enable hydroxylation, prior to treating the surface with APTES and GA.

In yet a further embodiment, the pre-treatment step includes plasma irradiation treatment with oxygen gas.

In an embodiment, the crown ether is a benzo-18-crown- 6. Typically, the crown ether compounds are carboxyl-benzo-18-crown-6 and 4-aminobenzo-18-crown-6.

In an embodiment, the step of treating the surface with APTES and GA further includes diluting APTES in ethanol and GA in deionized water, typically 2% (v/v) APTES and 0.1% (v/v) GA.

In an embodiment, the step of coating the APTES-GA surface with the crown-ether ionophore includes diluting the crown ether compound in methanol.

In an embodiment, the method further includes diluting the crown ether compound in methanol.

In another aspect, the present invention provides an optical sensor for detecting ions in a sample, comprising a resonant structure with an ionophore layer prepared with a crown ether compound, wherein when a targeted ion is in contact with the functionalized ionophore layer, the material index thereof changes due to absorption of the targeted ions therefore enabling the concentration of the targeted ion in the sample to be determined.

In one embodiment, the resonant structure comprises a pair of waveguides in the form of a sensing arm which is coated with the functionalized ionophore layer and a reference arm which includes a barrier coating over the functionalized ionophore layer (or is not coated with the functionalized layer). Typically, the resonant structure is formed in silicon or silicon oxide.

Typically, the channel widths of the waveguides range from 15 to 1000 nm and the lengths from 200 μm to 1 cm. Typically the thickness of the waveguides is around 220 nm.

In another embodiment, a light source is configured to emit light of at least one wavelength which is directed through said pair of waveguides after a predetermined exposure time thereby generating respective patterns which can be compared to determine the concentration of the targeted ion. Typically, the light source emits light wave within a wavelength range of 1500 to 1600 nm.

In one embodiment, the exposure time for detection of ions is 20 seconds and above.

In a further embodiment, the sensor includes a spectral interrogator for detecting and measuring the concentration of ions.

Typically, the targeted ion is K+.

In an embodiment, the concentration of the detected ion in the sample is derived based on the measurement of the resonant wavelength shift against time.

### BRIEF DESCRIPTION OF DRAWINGS

The invention will be more understood by reference to the description below taken in conjunction with the accompanying drawings herein:

FIG. 1A provides a schematic diagram of the binding mechanism of potassium ion with carboxyl-functionalized benzo-18-crown-6 in accordance with an embodiment of the present invention;

FIG. 1B provides a schematic diagram of the binding mechanism of potassium ion with 4-aminobenzo-18-crown-6 in accordance with an embodiment of the present invention;

FIG. 2A shows the top view of the sensor chip in accordance with an embodiment of the present invention;

FIG. 2B provides an example of the waveguide in accordance with an embodiment of the present invention;

FIG. 3 shows a flowchart for the method in accordance with an embodiment of the present invention;

FIG. 4 shows an example of wavelength shift with respect to material index changes, in accordance with an embodiment of the present invention;

FIG. 5A-FIG. 5B show examples of wavelength shift due to increased ions concentration

### DETAILED DESCRIPTION

In line with the above summary, the following description of a number of specific and alternative embodiments is provided to understand the inventive features of the present invention. It shall be apparent to one skilled in the art, however that this invention may be practiced without such specific details. Some of the details may not be described at length so as not to obscure the invention. For ease of reference, common reference numerals will be used throughout the figures when referring to the same or similar features common to the figures.

Embodiments of the invention are described by way of illustration. As will be realized, the invention is capable of other and different embodiments and its several details are capable of modifications in various respects, all without departing from the scope of the present invention. It should be noted that the drawings include a flow chart of how the method in accordance with the preferred embodiments can be carried out. Standard equipment or components may have not been illustrated since they are known in the art.

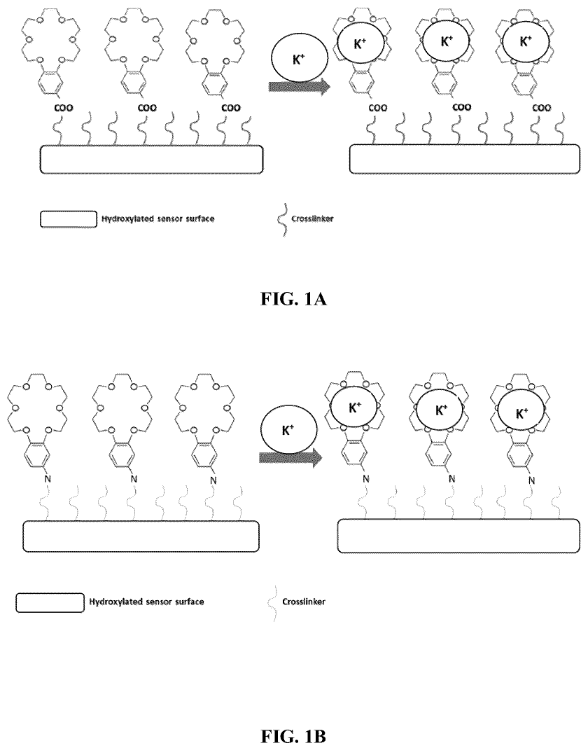

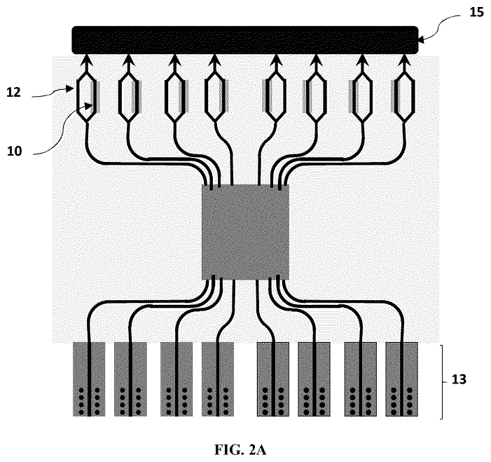

The present invention provides a potassium ion-selective optical sensor, comprising a functionalized ionophore layer prepared with a macrocyclic compound, more particularly an amino-and carboxyl-crown ether compound, such that, and as shown in FIG. 1, when the targeted ion is in contact with the amino-and carboxyl-crown ether functionalized layer, the material index of the functionalized layer changes due to absorption kinetics of the targeted ions on the functionalized layer, therefore enabling the detection of the potassium ion in a sample. Accordingly, the concentration of the ion in said sample may be derived by means measuring the resonant wavelength shift against time. In a preferred embodiment, the sensor is an optical sensor comprising a resonant surface and waveguide sections of different widths.

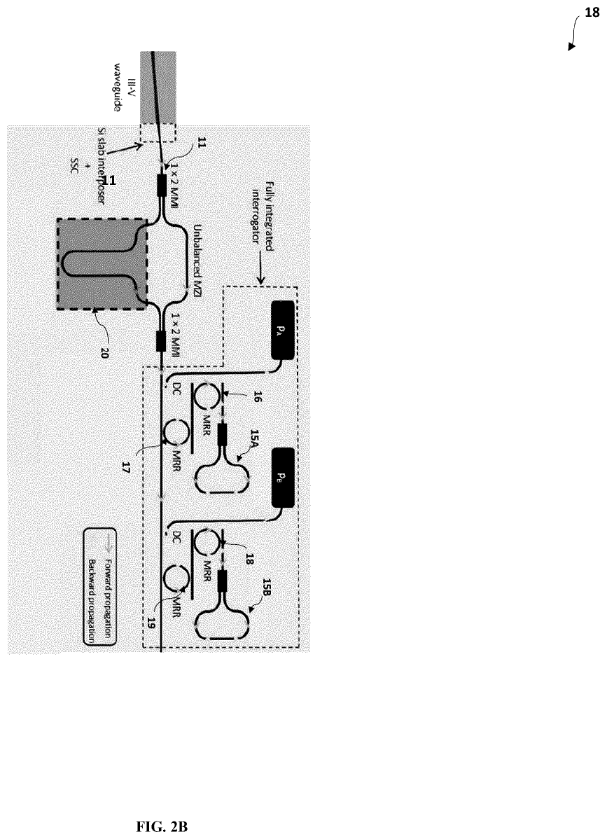

Now referring to FIG. 2A, a light source (13) projects a beam that passes through an input waveguide prior to splitting equally at a Y-junction, and then guided to propagate along a sensing exposed arm (10) and a reference arm (12) in the form of straight waveguides. In the preferred embodiment, the reference arm (12) is coated with a material that prevents the sensing of potassium ions therefore provides a reference wavelength pattern corresponding to a condition in which potassium ions is non-existent, while the sensing arm (10) is coated with the functionalized layer to detect potassium ions and provides a wavelength pattern corresponding to the concentration of potassium ions. The chemical reaction in the sensing arm (10) can be measured by interference intensity at an output waveguide which is then measured by an interrogator (15).

Accordingly, the sensor can be calibrated and optimized based on each different pair of channels having different characteristics to provide the greatest shift at a particular or selected wavelength. Advantageously, the comparison allows shifts of <500 pm to be measured accurately, corresponding to a sensitivity 10 ppb potassium ions.

In an embodiment, the functionalized layer may be provided on a surface in a waveguide-based sensor comprising a chip with Mach-Zehnder interferometer (MZI) configuration. An example of a MZI optical waveguide sensor with a silicon on insulator platform (SOI) comprising the functionalized layer of the present invention is shown in FIG. 2B. In this embodiment, the sensing section (20) comprises waveguides with a silicon oxide SiO₂ cladding, having a thickness of about 220 nm, width 15-1000 nm and length about 200 μm-1 cm. The coupling is a vertical grating coupler (11), preferably in transverse mode. The wavelength range is 1500-1600 nm, with a resolution measurement up to 1 picometer (pm). An interrogator (15) is connected to an output, in which the interrogator (15) may comprise of a pair of MMI-based reflectors (15A, 15B) and 2 pairs of micro-ring resonators (16,17, 18, 19). Suitably, the readout parameter is in phase or lambda.

In use, the following sequence may be observed when a test is implemented with the MZI system. First, a drop of distilled water is added as a blank sample on the surface of the MZI system. Second, the droplet of distilled water is removed from the surface. Third, a drop of solution having a known concentration of K⁺ is added on the surface. After 30 seconds to 2 minutes, the solution is removed from the surface. Then, a drop of distilled water is again added to the surface. Last but not least, the laser is swept from a wavelength of 1500 to 1600 nm, and the output of the sensor is measured.

The present invention further provides a method for fabricating or forming said ion-selective sensor for determining the presence of an ionic species in a sample. More specifically the present invention provides a method for fabricating a sensor for the detection of potassium (K⁺) ion, in which a functionalized ionophore prepared with an amino- and carboxyl-crown ether compound is coated on a surface of said sensor, such that it forms a layer on the surface of the sensor.

Generally, macrocyclic compounds such as crown ethers are known for their unique property of forming stable complexes with alkali metals due to the close fit of cation to the cavity and a nearly planar oxygen atoms around the central cation. The crown ethers compound synthesized in accordance with the present invention comprise moieties that are typically selective of the cation, i.e., potassium, therefore enabling the detection of such species in a sample.

In an embodiment of the present invention, the binding mechanism, hence strongly bound complexes and ion-dipole interaction of potassium ions to the crown ether compound is created by inclusion of the cation into the cavity where the electrostatic interaction with the partially negative charges on the oxygen atoms.

In a preferred embodiment, the method for fabricating an ion-selective sensor comprises the following steps: treating a surface of the sensor with (3-aminopropyl)triethoxysilane (APTES) followed by glutaraldehyde (GA); coating the APTES-GA surface with a crown ether-ionophore to create a functionalized layer; wherein when the ionophore functionalized layer is in contact with the targeted ion, the material index of the functionalized crown-ether ionophore layer changes therefore enabling the detection of the targeted ion in a sample.

Typically, the crown ether ionophore includes a benzo-18-crown-6, and more particularly carboxyl-benzo-18-crown-6 and 4-aminobenzo-18-crown-6; however, it is anticipated that other benzo crown compounds and derivatives with moieties that are selective or capable of selectively binding potassium ions may be used in the method.

The method includes using non-toxic solvents such as water, ethanol and methanol in the steps of preparing the functionalized ionophore layer.

In the preferred embodiment, treating the surface with (3-aminopropyl) triethoxysilane (APTES) and glutaraldehyde (GA) initiates chemical crosslinking reaction for covalent binding which is then followed by immobilization of carboxyl-benzo-18-crown-6 and 4-aminobenzo-18-crown-6 ionophore onto a surface of the sensor.

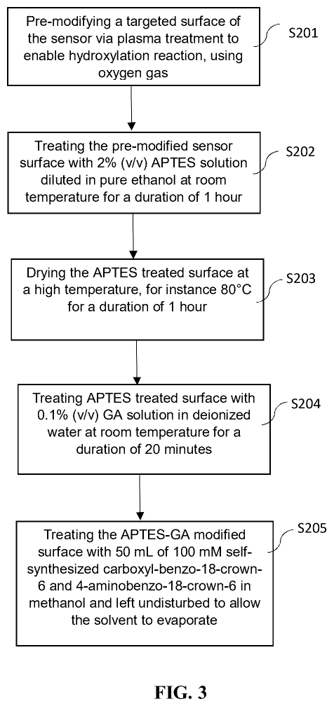

FIG. 3 provides a flow chart depicting the steps of the method for fabricating the ion-selective optical sensor, the method comprising: pre-modifying a targeted surface of the sensor via plasma treatment to alter the surface and enable hydroxylation reaction, using oxygen gas (S201). After plasma treatment, the pre-modified sensor surface is treated with 2% (v/v) APTES solution diluted in pure ethanol at room temperature for a duration of 1 hour (S202), followed by drying the APTES treated surface at a high temperature, for instance 80° C. for a duration of 1 hour (S203). The dried APTES treated surface is then treated with 0.1% (v/v) GA solution in deionized water at room temperature for a duration of 20 minutes (S204). The APTES-GA modified surface is further treated with 50 mL of 100 mM self-synthesized carboxyl-benzo-18-crown-6 and 4-aminobenzo-18-crown-6 ionophore in methanol and left undisturbed to allow the solvent to evaporate (S205).

The functionalized ionophore layer is heterogeneously integrated with the resonant surface of the sensor, such that the resonant condition of the sensor shifts accordingly to the change of material index due to the absorption of the ions, therefore the concentration of the ions in the sample can be measured and quantified by detecting the shift in resonant wavelength curves against time curves. The same technique may be used to train artificial neural network for deriving the concentration of the ions.

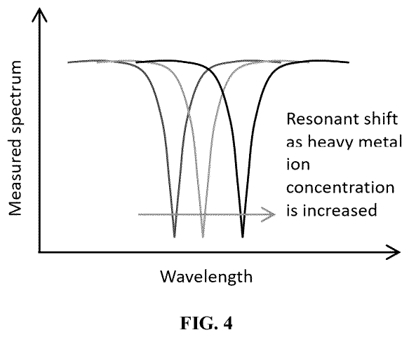

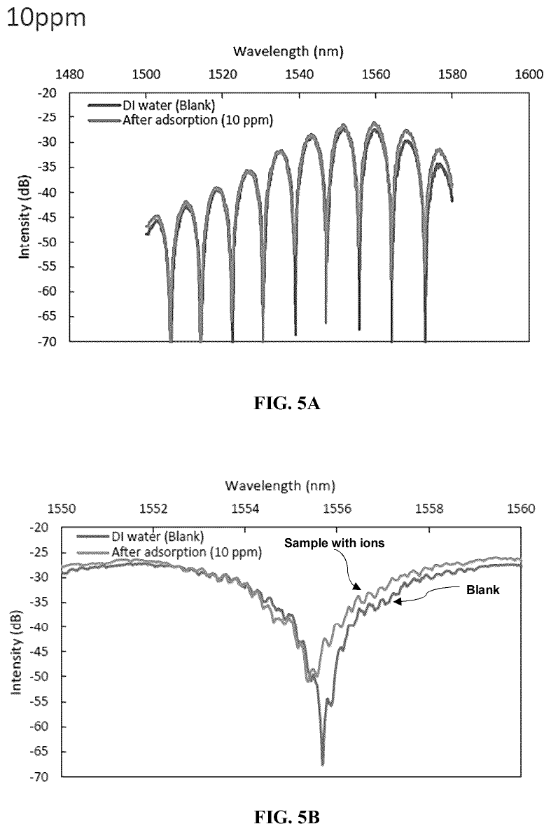

An example of wavelength shifts of the optical sensor integrated with the functionalized layer due to change of material index is shown in FIG. 4. Accordingly, as the layer absorbs the ion, the change in the material index causes a shift in the wavelength, which facilitates in measuring or quantifying the ion concentration in the sample. This is clearly shown in FIG. 5A and FIG. 5B, in which the wavelength shifts as the functionalized layer absorb more ions from a sample. The presence of potassium ions can be detected within a predetermined exposure time for example within 30 to 120 seconds, in which the change or wavelength shift does not substantially change further after 120 seconds.

In a preferred embodiment, the targeted surface of the sensor includes a resonant surface or structure, in which the functionalized ionophore prepared in accordance with the method may be applied on resonant structures or a resonant surface of a sensor such as, Mach-Zehnder interferometers, mirroring resonators or on an integrated photonics platform i.e., III-V, silicon photonics.

The concentration of the potassium ions may be tuned to optimise the detection range, in which higher detection range of the ions may be achieved by increasing the concentration of the ions and sensitivity of the sensor. In an embodiment, the sensor can detect and measure ion concentrations from 10 ppb up to 200 ppm.

In contrast to conventional methods or devices such as ICP-MS/OES that require complicated sample pre-processing and highly skilled personnel to operate, the integration of the functionalized with the resonant surface of the sensor enables a less complicated, on-site, in-situ measurement to be conducted, as the sensor may be realized in compact and portable form.

The sensor may be mass produced by varying the concentration of the crown ethers and their linkers onto a single sensor area, hence providing a sensor that could be deployed in any system or environment.

While the invention has been described as required in terms in preferred embodiments and specific operating ranges and conditions, those skilled in the art will appreciate that the invention described herein is susceptible to variations and modifications other than those specifically described.

1. A method for forming an optical ion-selective sensor, the method comprising the steps of:
treating a resonant structure on the surface of the sensor with (3aminopropyl)triethoxysilane (APTES) followed by glutaraldehyde (GA); and
coating the APTES-GA surface with a crown-ether ionophore to create a functionalized layer;
wherein when the functionalized layer is in contact with a potassium ion in a sample, a complex is formed between the crown ether and the potassium ion therefore changing a material index of the functionalized layer, the change corresponding to the concentration of the potassium ion in the sample.

2. (canceled)

3. The method according to claim 1, wherein changing a material index shifts the resonant condition of the layer therefore enabling the measurement of ion concentration.

4. (canceled)

5. The method according to claim 1, wherein the method further includes a pretreatment step to modify the surface of the sensor to enable hydroxylation, prior to treating the surface with APTES and GA.

6. The method according to claim 5, wherein the pre-treatment step includes plasma irradiation treatment with oxygen gas.

7. The method according to claim 1, wherein the crown ether ionophore includes a benzo 18-crown-16.

8. The method according to preceding claim 1, wherein the crown ether ionophore includes carboxyl-benzo-18-crown-6 and 4-aminobenzo-18-crown-6.

9. The method according to claim 1, wherein the step of treating the surface with APTES and GA further includes diluting APTES in ethanol and GA in deionized water.

10. The method according to claim 1, wherein the step of coating the APTES-GA surface with the crown ether ionophore includes diluting the crown ether compound in methanol.

11. The method according to claim 1, wherein the surface is treated with 2% (v/v) APTES solution diluted in ethanol and 0.1% (v/v) GA solution in deionized water.

12. An optical sensor for detecting ions in a sample, comprising a resonant structure with a functionalized ionophore layer prepared with a crown ether compound, wherein when a targeted ion is in contact with the functionalized ionophore layer, the material index thereof changes due to absorption of the targeted ions, therefore enabling the concentration of the targeted ion in the sample to be determined.

13. The optical sensor according to claim 12, wherein the resonant structure further comprises waveguides to form a sensing arm which is coated with the functionalized ionophore layer and a reference arm which includes a barrier coating over the functionalized ionophore layer.

14. The optical sensor according to claim 13, wherein the channel widths of the waveguides range from 15 to 1000 nm, and the lengths from 200 μm to 1 cm.

15. The optical sensor according to claim 13 wherein a light source is configured to emit light of at least one wavelength which is directed through said pair of waveguides after a 30 predetermined exposure time, thereby generating respective patterns which can be compared to determine the concentration of the targeted ion.

16. The optical sensor according to claim 15, wherein the light source emits light within a wavelength range of 1500 to 1600 nm.

17. The optical sensor according to claim 12 wherein the concentration of the detected ion in the sample is derived based on the measurement of the resonant wavelength shift against time.

18. The optical sensor according to claim 12, wherein the exposure time for detection of ions is seconds and above.

19. The optical sensor according to claim 12, wherein the sensor further includes a spectral interrogator for detecting and measuring the concentration of ions.

20. The optical sensor according to claim 12, wherein the targeted ion is K+.

21. The optical sensor according to claim 12, wherein the crown ether ionophore layer includes carboxyl-benzo-18-crown-6 and 4-aminobenzo-18-crown-6.

22. (canceled)
